(19) United States
(12) Patent Application Publication
(10) Pub. No.: US 2010/0292797 A1
(43) Pub. Date: Nov. 18, 2010

(54) Facet joint implant

(75) Inventors: Stephan Lindner, Wurmlingen (DE); Alexander Haas, Donaueschingen (DE); Ulrike Kettenberger, Trostberg (DE)

Correspondence Address: Lipsitz & McAllister, LLC, 755 MAIN STREET, MONROE, CT, 06468, US

(73) Assignee: Aesculap AG, Tuttlingen (DE)

(21) Appl. No.: 12/800,138

(22) Filed: May 6, 2010

(30) Foreign Application Priority Data
May 13, 2009 (DE) 10 2009 021 134.9

(51) Int. Cl. A61F 2/44

(52) U.S. Cl. 623/17.11

(57) ABSTRACT

In order to improve the process of inserting a facet joint implant comprising two, flat joint surface replacement elements abutting each other face-to-face and in order to control the movement of the joint surface replacement elements relative to each other, it is proposed that both joint surface replacement elements should carry at least one arm which emerges in the plane of the joint surface replacement element from said element and projects therefrom, and in that in each case two arms of the two joint surface replacement elements which extend adjacent to one another in a pair are connected together at the end thereof remote from the joint surface replacement element.

This application claims the benefit of German Patent Application No. 10 2009 021 134.9 filed on May 13, 2009.

The present disclosure relates to the subject matter disclosed in German patent application DE 10 2009 021 134.9 of May 13, 2009, which is incorporated herein by reference in its entirety and for all purposes.

### BACKGROUND OF THE INVENTION

The invention relates to a facet joint implant comprising two flat joint surface replacement elements abutting each other face-to-face.

The facet joints represent one significant cause for the occurrence of back pain. A remedy for this can be obtained by replacing the entire facet joint or by the replacement of the facet joint surfaces after removal of the pain-inducing articular capsules and cartilage layers.

It is known to permanently connect the facets of neighbouring vertebral bodies rigidly together and thus prevent the movement of the two vertebral bodies in the facet joint but, as a consequence thereof, the patient's movements will be restricted.

It has also known to replace the joint surfaces of the facet joint by joint surface replacement elements, in general, these are thin platelets which are placed on the facets and connected thereto and which abut face-to-face against the neighbouring facet or the joint surface replacement surface on the neighbouring facet. It is difficult however to implant these facet joint replacement surfaces. Moreover, there is a need to limit the movement of the mutually engaging joint surface replacement elements relative to each other. Finally, it is also of importance that the size of an implant of this type should not be too great so that the implant can be placed on the location for the implantation process through small access openings.

The object of the invention is to provide a facet joint implant of the above type in such a way that the aforesaid advantages can be obtained.

### SUMMARY OF THE INVENTION

In accordance with the invention, this object is achieved in the case of a facet joint implant of the type described hereinabove in that both joint surface replacement elements carry at least one arm which emerges in the plane of the joint surface replacement element from said element and projects therefrom, and in that in each case two arms of the two joint surface replacement elements which extend adjacent to one another in a pair are connected together at the ends thereof remote from the joint surface replacement element.

Due to the connection of the two joint surface replacement elements by means of the mutually adjacent arms projecting from the joint surface replacement elements, the implant then becomes a single component which can be implanted as such in a relatively simple manner. Moreover, the arms limit the movement of the joint surface replacement elements relative to each other so that the latter—mutually abutting—elements cannot be displaced arbitrarily with respect to one another, but rather, there is a restriction on the movement thereof due to the two interconnected arms.

In a first preferred embodiment, provision is made for the two mutually adjacent arms extending from the two joint surface replacement elements to be connected to one another in one-piece manner.

Furthermore, provision may be made for the arm emerging from a joint surface replacement element to be connected to the joint surface replacement element in one-piece manner

In accordance with a preferred embodiment, the arm is in the form of a band.

It is advantageous here if the band and the joint surface replacement element extend in a plane in the region of emergence of the band.

Furthermore, it can be advantageous for the band and the joint surface replacement element to be of equal thickness in the region of emergence.

In particular, the two arms can be formed by a continuous band.

The band can form a U-shaped loop.

In another preferred embodiment, provision is made for the band to have an undulating formation in the region where the two arms join so that a spring effect is thereby produced.

In a preferred embodiment, the joint surface replacement element is formed by a widened end section of an arm.

In a preferred embodiment, a spring element which connects the two arms together resiliently is arranged in the region of connection of the two arms.

The spring element can, for example, be formed by an elastic section of the arms themselves or else by means of a separate component that is arranged between the arms.

In another embodiment, provision is made for the two arms to be connected to one another in hinge-like manner in the region of connection. The type of hinge may vary, it may be an actual hinge incorporating a hinge pin, a film hinge or even a hinge that is pivotal in every direction and could be implemented as a ball joint for example.

Furthermore, it is possible for the two arms to be connected together in the region of connection thereof such that they are displaceable relative to each other in a guidance means.

The guidance means can, for example, be in the form of an elongate slot in one arm through which a narrow section of the other arm can pass. In another embodiment, the guidance means is in the form of a telescopic guidance means.

The width and/or the thickness of the arms can vary over the length thereof. It is thereby possible to adjust the stiffness of the arms to a preferred value and thus produce the desired limitation on the movement of the joint surface replacement elements, and in particular, to restrict such movement. It is also possible to produce such an effect by arms of differing lengths.

In a preferred embodiment, provision is further made for there to be arranged, on the two joint surface replacement elements, a plurality of arms which emerge from each of the joint surface replacement elements in different directions, the ends of the arms remote from the joint surface replacement elements being connected together in respective pairs. In particular, provision may be made for three pairs of arms which emerge from each of the joint surface replacement elements substantially perpendicularly to each other to be arranged on the two joint surface replacement elements.

The following description of preferred embodiments of the invention taken in conjunction with the drawing will serve to provide a more detailed explanation.

### BRIEF DESCRIPTION OF THE DRAWINGS

FIG. 1 shows a perspective view of two vertebral bodies with a facet joint implant inserted into a facet joint;

FIG. 2 is an enlarged perspective view of a first embodiment of a facet joint implant;

FIG. 3 is a plan view of the facet joint implant depicted in FIG. 2;

FIG. 4 is a view of the implant depicted in FIGS. 2 and 3 in the direction of the arms connecting the facet joint replacement elements;

FIG. 5 is a view similar to FIG. 2 of a further embodiment of a joint implant;

FIG. 6 is a plan view of the joint implant depicted in FIG. 5;

FIG. 7 is a side view of the joint implant depicted in FIGS. 5 and 6;

FIG. 8 is a view similar to FIG. 5 of a further embodiment of a joint implant comprising three pairs of arms;

FIG. 9 is a plan view of the joint implant depicted in FIG. 8;

FIG. 10 is a sectional view along the line 10-10 in FIG. 9;

FIG. 11 is a perspective view of a joint implant having helically-configured arms in the region of connection of the two arms;

FIG. 12 is a perspective view of a joint implant comprising an elastic body between the two arms;

FIG. 13 is a perspective view of a joint implant comprising arms which are connected together in hinge-like manner;

FIG. 14 is a perspective view of a joint implant comprising arms which are connected together by a slot-guidance arrangement;

FIG. 15 is a perspective view of a joint implant comprising three strip-like arms in the form of loops;

FIG. 16 is a perspective view of a joint implant comprising two arms which are guided in telescopic manner relative to each other;

FIG. 17 is a perspective view of a joint implant comprising two band-like arms which are connected together by an undulating region and

FIG. 18 is a joint implant comprising two arms which are guided in telescopic manner relative to each other.

### DETAILED DESCRIPTION OF THE INVENTION

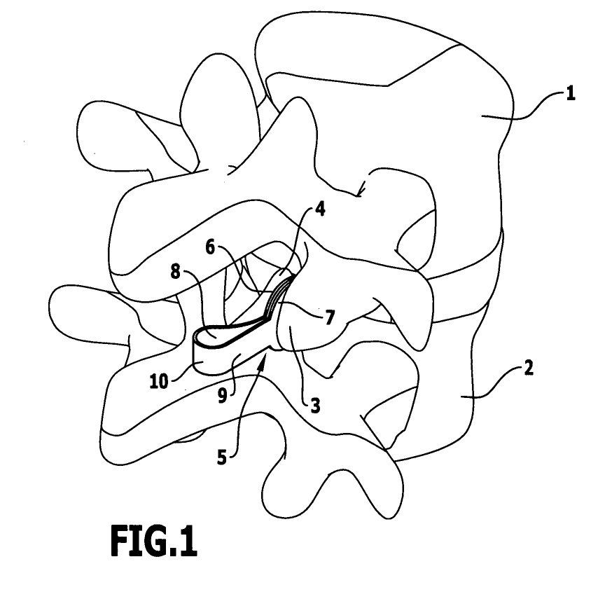

Two vertebral bodies 1, 2 which are arranged adjacent to one another and comprise respective facets 3 and 4 that rest upon one another are illustrated in FIG. 1. A facet joint implant 5, which comprises two flat joint platelets 6, 7 abutting each other face-to-face, is inserted between the facets 3, 4—if necessary after removal of the joint capsule and/or the cartilage layer of the facets. These joint platelets 6, 7 are fixed in an appropriate manner to the facets 3, 4, for example, by means of projections arranged on the outer surfaces of the joint platelets 6, 7, by means of a tissue glue or with the aid of other known means. The joint platelets 6, 7 are thereby firmly connected to the facets 3, 4 and form a joint replacement surface since the joint platelets 6, 7, which abut each other face-to-face, can be displaced with respect to each other and thereby move with respect to one another substantially parallel to the plane of the joint platelets 6, 7.

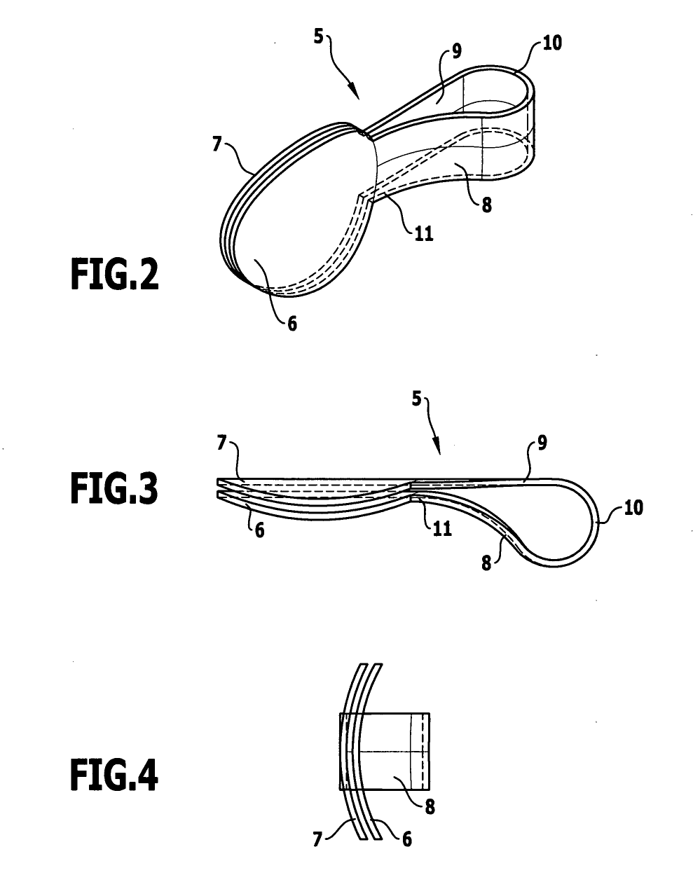

In the embodiment illustrated in FIGS. 2 to 4, band-like arms 8, 9, which are connected together in one-piece manner, emerge from the two joint platelets 6, 7 in the plane of the joint platelets i.e. the arms 8 and 9 form a continuous band which interconnects the two joint platelets 6, 7. In this case, the band forming the two arms 8, 9 is arranged to be equally thick and equally wide over its entire cross section, and moreover, the thickness thereof substantially corresponds to the thickness of the joint platelets 6, 7. This band connecting the two joint platelets 6, 7 is distorted when the two joint platelets 6, 7 are displaced with respect to one another and thereby restricts the relative movement of the two joint platelets 6, 7 as a result of the restoring forces thereby arising. The degree of restriction is dependent on the width, the length and also the thickness of the arms 8, 9 and can be affected by appropriate choice of the magnitudes thereof.

In the embodiment illustrated in FIGS. 2 to 4, the joint platelets 6, 7 as well as the interconnecting arms 8, 9 in the form of a continuous band are formed in one-piece manner and consist for example, of a body-compatible synthetic material such as polyetheretherketone.

The joint implant thereby forms a component which, although of course having a certain extent in the longitudinal direction, is only of very small size in the direction transverse thereto so that this implant can be introduced into the body in a simple manner through narrow body openings and can be placed in a simple manner between two facets 3, 4 in such a way that the loop formed by the arms 8, 9 is spaced from the vertebral bodies 1, 2, as is illustrated in FIG. 1.

In the embodiment depicted in FIGS. 2 to 4, the joint platelets 6, 7 have approximately the form of a circle whereby the arms 8, 9 emerge from the joint platelets 6, 7 in the radial direction, the width of the arms 8, 9 is less than the diameter of the joint platelets 6, 7, for example, said width may be in an order of magnitude of between 30 and 60% of the diameter of the joint platelets 6, 7.

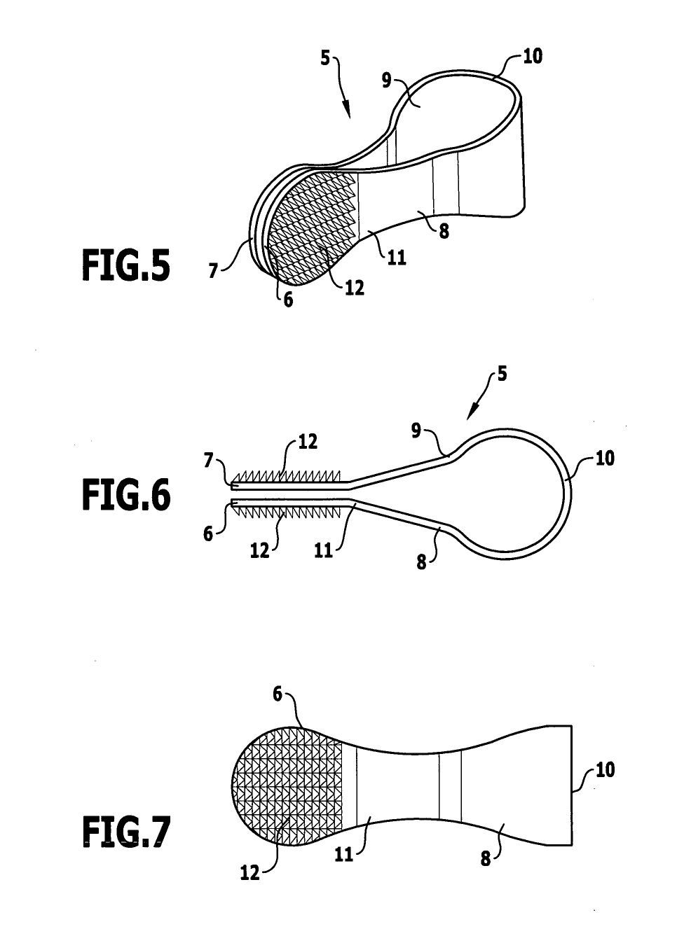

In the embodiment depicted in FIGS. 5 to 7, which is of substantially identical construction and therefore bears the same reference symbols for equivalent parts, the width of the arms 8, 9 varies over the length thereof, said arms being wider in the region of connection 10 remote from the joint platelets 6, 7 than in a region of transition 11 towards the joint platelets 6, 7, whilst the arms 8, 9 become continuously wider approaching the joint platelets 6, 7 so that the respective joint platelets 6, 7 can be regarded as being a widening of the arms 8, 9. In the embodiment depicted in FIGS. 5 to 7, there is also an illustration of projections 12 which are located on the outer surface of the joint platelets 6, 7 and with the aid of which the joint platelets 6, 7 are connected to the facets 3, 4 when they are placed thereon and the projections 12 entrench themselves into the bone material of the facets 3, 4. Projections 12 of this type can be provided in each of the embodiments, although this is not illustrated in all the drawings for reasons of clarity.

In the case of the joint implants depicted in FIGS. 2 to 7, the two joint platelets 6, 7 are each provided with one arm 8, 9 so that the two joint platelets 6, 7 are connected together by one pair of arms.

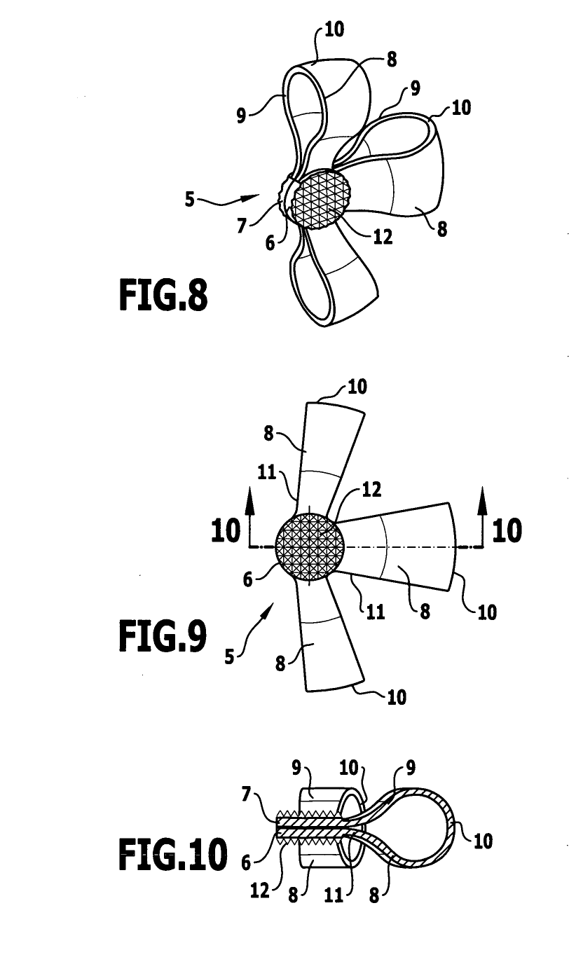

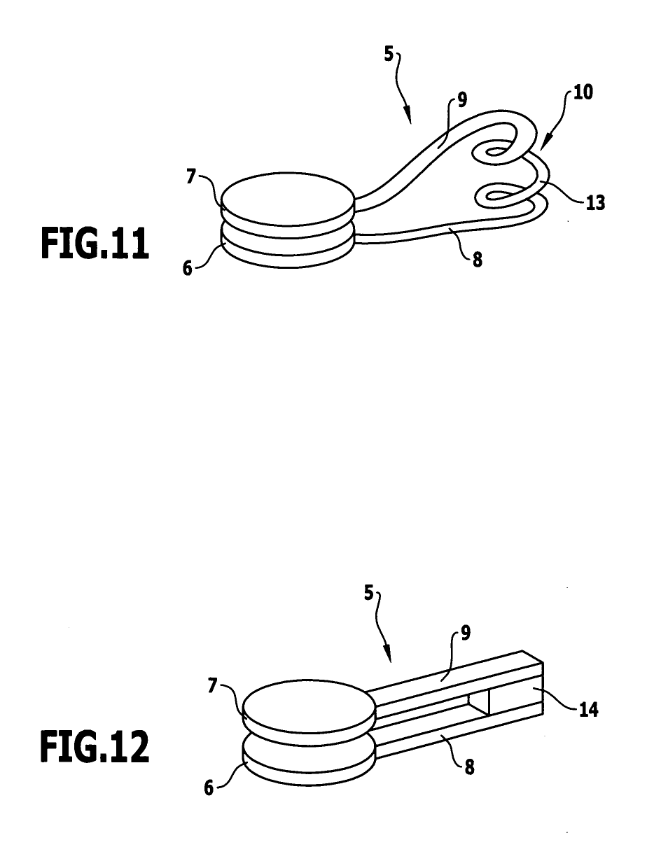

In contrast thereto, provision is made for the joint implant depicted in FIGS. 8 to 10, which is again of similar construction and bears the same reference symbols for the mutually corresponding parts, to have three pairs of arms of this type which are angularly displaced by approximately 90° over the periphery of the joint platelets 6, 7 so that the three pairs of arms thus extend approximately perpendicularly to each other. The number of these pairs of arms could also be selected to be different and it is also possible for the angular displacement of these pairs of arms to be different. For example, more than three pairs of arms could be used, these then including an angle therebetween which is smaller than 90°.

Whereas in the embodiments depicted in FIGS. 2 to 10 the two arms 8, 9 of a pair of arms are in the form of parts of a continuous band which emanates from the two joint platelets 6, 7 and forms a loop, it is also possible to have differing arrangements, for example, the two joint platelets 6, 7 could be connected together by two rod-shaped arms 8 and 9 which merge into one another in one piece manner in the form of a helix 13 in the region of connection 10 (FIG. 11). Thus, due to the self-elasticity of the material of the arms 8, 9, this then results in the formation of a spring element which connects the two arms 8, 9 together in the region of connection 11.

In the embodiment depicted in FIG. 12, such a spring element between the two arms 8, 9 is implemented in that the two arms 8, 9 are connected together in the region of connection 10 by means of a connecting block 14 consisting of an elastomeric material. This material could be a hydrogel or silicone for example.

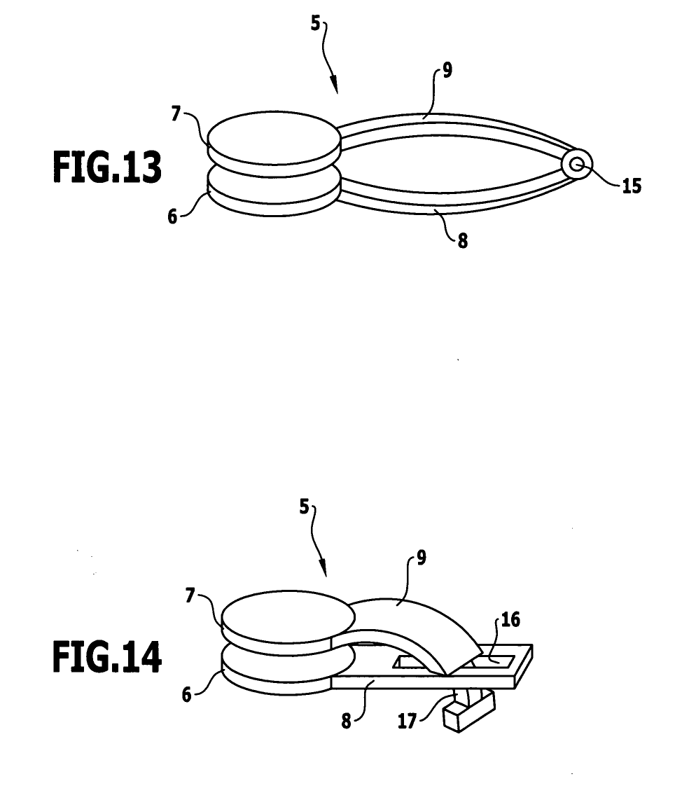

In FIG. 13, there is illustrated a joint implant in which the two rod-shaped arms 8, 9 are connected together in articulated manner in the region of connection 10 by means of a hinge 15, hereby, this could be a hinged joint incorporating a hinge pin, although it would also be possible for a hinged joint in the form of a film hinge to be provided in this region or for the joint to be in the form of a ball joint.

In the embodiment depicted in FIG. 14, both arms 8, 9 are in the form of a band, one of the two arms incorporating an elongate through slot 16 through which the other arm 9 can pass. In the region where it passes through, this arm 9 comprises a narrower bearing section 17 so that the arm 9 is displaceable relative to the arm 8 along the elongate slot 16, although it is prevented from leaving the elongate slot 16 due to the fact that the arm 9 is of greater width on both sides of the narrower bearing section 17.

In the embodiment depicted in FIG. 15, use is made of arms 8, 9 which have a bar-like cross section where the width thereof is only slightly greater than the thickness. Here, the arms have a continuous one-piece construction and are connected to one another in one-piece manner and, in similar manner to the embodiment depicted in FIGS. 8 to 10, three mutually transversely extending pairs of arms are provided in the embodiment depicted in FIG. 15.

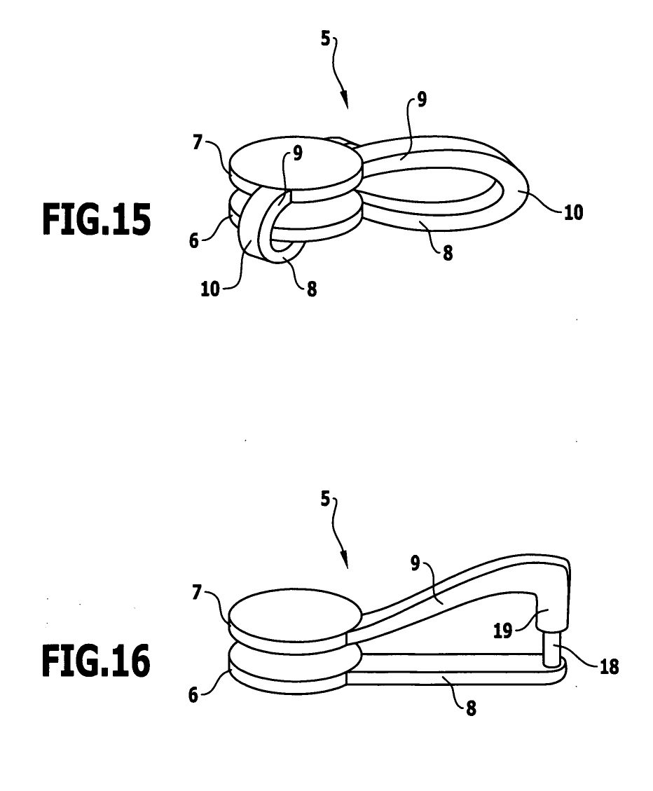

In the case of the embodiment depicted in FIG. 16, one arm 8 carries a bearing pin 18 at its free end, said pin projecting transversely relative to the longitudinal extent of the arm and being surrounded by a bearing sleeve 19 which is open relative to the bearing pin 18 and which is arranged on the other arm 9 and projects transversely relative to the longitudinal direction of said other arm. This thereby results in a telescopic guidance arrangement for the bearing pin 18 and the bearing sleeve 19, the two arms being connected in mutually displaceable manner by this guidance arrangement. A similar arrangement is realized in the joint implant depicted in FIG. 18, wherein provision is made for one arm 8 to have a similar bearing sleeve 19 into which a bent section 20 of the other arm 9 is insertable, this arm 9 carrying at its free end a bearing plate 21 which guides the bent section 20 within the bearing sleeve 19 so that a telescopic arrangement for the guidance of the two arms relative to each other also arises here. At the upper edge thereof, the bearing sleeve 19 comprises a radially inwardly protruding flange 22 which serves to limit the movement of the bearing plate 21 so that unwanted separation of the two arms 8 and 9 is impossible.

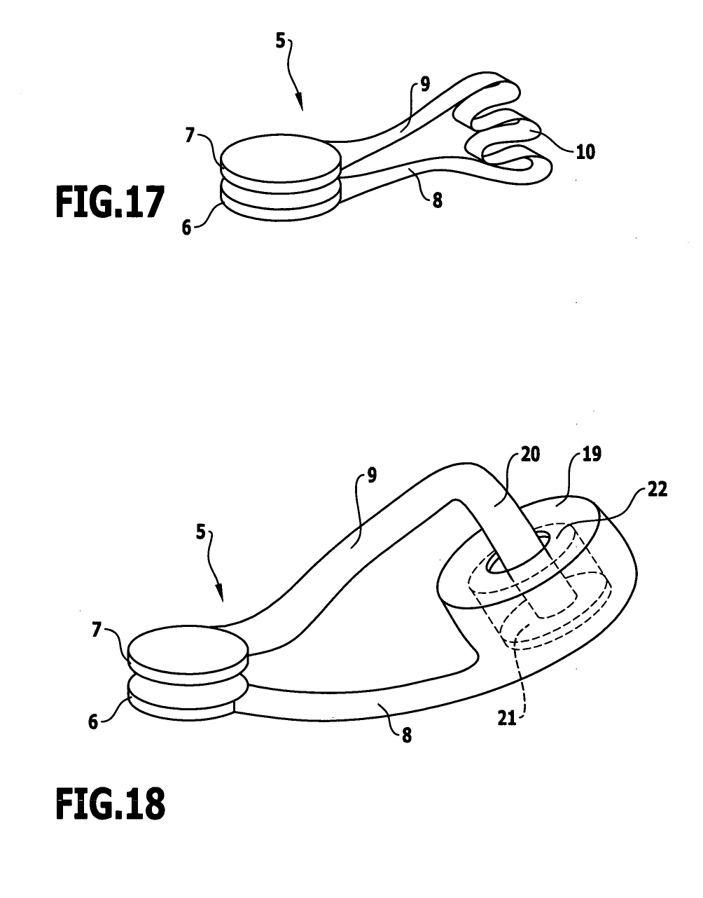

In the embodiment depicted in FIG. 17, the two arms 8, 9 are band-like in a similar manner to the embodiment depicted in FIGS. 2 to 4 although they are not connected together in one-piece manner by a simple loop, but rather, the band forming the two arms is bent back and forth several times in the region of connection 10 in an undulating manner so that a springy effect assisted also by the self-elasticity of the band thereby arises.

1. A facet joint implant comprising two flat joint surface replacement elements abutting each other face-to-face, wherein both joint surface replacement elements carry at least one arm which emerges in the plane of the joint surface replacement element from said element and projects therefrom, and in each case two arms of the two joint surface replacement elements which extend adjacent to one another in a pair are connected together at the ends thereof remote from the joint surface replacement elements.

2. A facet joint implant in accordance with claim 1, wherein the two mutually adjacent arms extending from the two joint surface replacement elements are connected to one another in one-piece manner.

3. A facet joint implant in accordance with claim 1, wherein the arm emerging from a joint surface replacement element is connected to the joint surface replacement element in one-piece manner.

4. A facet joint implant in accordance with claim 1, wherein the arm is in the form of a band.

5. A facet joint implant in accordance with claim 4, wherein the band-like arm and the joint surface replacement element extend in a plane in the region of emergence of the arm.

6. A facet joint implant in accordance with claim 4, wherein the arm and the joint surface replacement element are of equal thickness in the region of emergence of the arm.

7. A facet joint implant in accordance with claim 4, wherein the two arms are formed by a continuous band.

8. A facet joint implant in accordance with claim 7, wherein the continuous band forms a U-shaped loop.

9. A facet joint implant in accordance with claim 7, wherein the band has an undulating formation in the region of connection of the two arms.

10. A facet joint implant in accordance with claim 1, wherein the joint surface replacement implants are formed by a widened end section of an arm.

11. A facet joint implant in accordance with claim 1, wherein a spring element is arranged in the region of connection of the two arms.

12. A facet joint implant in accordance with claim 11, wherein the spring element is formed by an elastic section of the arms themselves.

13. A facet joint implant in accordance with claim 11, wherein the spring element is formed by a separate component which is arranged between the arms.

14. A facet joint implant in accordance with claim 1, wherein the two arms are connected together in the region of connection in hinge-like manner or in the form of a ball joint.

15. A facet joint implant in accordance with claim 3, wherein the two arms are connected together in the region of connection thereof in mutually displaceable manner in a guidance means.

16. A facet joint implant in accordance with claim 15, wherein the guidance means is an elongate slot in one arm through which a narrow section of the other arm passes.

17. A facet joint implant in accordance with claim 15, wherein the guidance means is a telescopic guidance means.

18. A facet joint implant in accordance with claim 1, wherein the width and/or the thickness of the arms varies over the length thereof.

19. A facet joint implant in accordance with claim 1, wherein a plurality of arms which emerge from each of the joint surface replacement elements in different directions are arranged on the two joint surface replacement elements, the ends of the arms remote from the joint surface replacement elements being connected together in respective pairs.

20. A facet joint implant in accordance with claim 19, wherein three pairs of arms which emerge from each of the joint surface replacement elements substantially perpendicularly to each other are arranged on the two joint surface replacement elements.

21. A facet joint implant in accordance with claim 2, wherein the arm emerging from a joint surface replacement element is connected to the joint surface replacement element in one-piece manner.

22. A facet joint implant in accordance with claim 2, wherein the arm is in the form of a band.

23. A facet joint implant in accordance with claim 3, wherein the arm is in the form of a band.

24. A facet joint implant in accordance with claim 5, wherein the two arms are formed by a continuous band.

25. A facet joint implant in accordance with claim 24, wherein the continuous band forms a U-shaped loop.
